(19) United States
(12) Patent Application Publication
(10) Pub. No.: US 2004/0175762 A1
(43) Pub. Date: Sep. 9, 2004

(54) Screening system based on expression of abcg2 half transporter protein

(76) Inventors: Csilla Ozvegy, Budapest (HU); Gergely Szakacs, Budapest (HU); Andras Varandi, Budapest (HU); Zoltan Nagy, Budapest (HU)

Correspondence Address: WELSH & KATZ, LTD, 120 S RIVERSIDE PLAZA, 22ND FLOOR, CHICAGO, IL, 60606, US

(21) Appl. No.: 10/469,562

(22) PCT Filed: Mar. 4, 2002
(86) PCT No.: PCT/HU02/00015

(30) Foreign Application Priority Data
Mar. 2, 2001 (HU) P0100947

(51) Int. Cl. G01N 33/53; G01N 33/567; C12N 5/06

(52) U.S. Cl. 435/7.2; 435/348

(57) ABSTRACT

The overexpression of an ABC half-transporter, ABCG2 (MXR/BCRP) transporter causes multidrug resistance in tumors. The present invention is based on the in vitro expression of the ABCG2 protein in insect cells as an expression system free of other, closely related, functional ABC transporters. In particular, the invention is related to a method for testing drugs for their effect on ABCG2 protein, method for the expression of active ABCG2, isolated ABCG2 homooligomers, preferably homodimers and uses thereof for testing drugs. The invention is further related to cell membrane preparations and cells, preferably insect cells comprising active ABCG2 and reagent kits for testing drugs for their effect on an ABCG2 half transporter protein. The invention is also related to a method for identifying ABCG2 activity in a biological sample, utilizing its substrate/inhibitor specificity.

The present invention is related to the in vitro expression of the ABCG2 protein in insect cells and application of this expression system for testing the interactions of various pharmacological agents and toxic materials with this protein. In particular, the invention is related to a method for testing drugs for their effect on ABCG2 protein, isolated ABCG2 homooligomers, insect cell membrane preparations comprising active ABCG2 and uses thereof, reagent kits for testing drugs and a method for identifying ABCG2 activity in a biological sample, utilizing its substrate/inhibitor specificity.

The multidrug resistant phenotype of malignant cells is the main obstacle in the chemotherapy treatment of patients suffering from cancer. It has been documented by numerous studies that the overexpression of multidrug transporters belonging to the ABC protein superfamily cause resistance of cancer cells by extruding most of the chemotherapeutic compounds currently used in treatment.

The two major ABC proteins involved in multidrug resistance are the well-characterized MDR1/Pgp (P-glycoprotein) and MRP1 (multidrug resistance associated protein). A third member of this group had recently been identified; the ABCG2 multidrug transporter. The protein is the product of the abcg2 gene

The expression of the ABCG2 multidrug transporter was detected in various cell lines that show multiple drug resistance—without the overexpression of MDR1/Pgp or MRP1—in e.g. breast cancer [Miyake et al., 1999; Ross et al., 1999; Yang et al., 2000], ovarian carcinoma [Maliepaard et al., 1999], colon carcinoma [Ross et al., 1999], leukemia [Ross 2000], Ehrlich ascites tumor cell lines [Nielsen et al, 2000], etc.) It was also shown that ABCG2 is overexpressed in the placenta [Allikmets et al., 1998].

Multiple experiments confirmed that ABCG2 confers multidrug resistance by actively extruding the cytotoxic compounds from the cells where it is overexpressed [Litman et al., 2000]. Bcrp1, the mouse homologue of ABCG2 was shown to confer resistance against mitoxantrone, topotecan and doxorubicin in mouse fibroblast cells [Jonker et al., 2000].

The characteristic domain of proteins belonging to the ABC transporter-family is the ATP Binding Casette (ABC) or nucleotide binding domain (NBD). ABC is responsible for nucleotide binding and hydrolysis, which provides energy for transport. The transmembrane domain (TIM), also found in ABC transporters, provides the interaction with the transported substrates. Human ABC transporters contain either one (ABC half-transporters) or two of the ABCs and TMDs. Since two ABCs and two TMDs are needed for a functional transporter, the half-transporters form dimers. In most of the human ABC transporters the ABC domain is found C-terminal to the TMD. The domain arrangement of members of the ABCG- or white-subfamily deviates from this general architecture.

The ABCG2-multidrug transporter, a 655 amino acid membrane protein, belongs to the ABCG/white subfamily. Members of the subfamily are ABC half-transporters with unique domain arrangement: they contain one ABC and one TMD where the ABC precedes (i.e. N-terminal to) the TMD (see FIG. 1/B.). The product of the Drosophila white gene (a homologue of ABCG2) forms a heterodimer with the brown or scarlet ABC-half transporters and transports guanine or tryptophan, respectively [Pepling and Mount, 1990; Dreesen et al., 1988; Tearle et al., 1989]. Other identified members of the human white subfamily are ABCG1 (ABC8), ABCG5 and ABCG8 [Berge et al., 2000]. ABCG1 is involved in the cholesterol and phospholipid transport of macrophages [Klucken et al., 2000]; it is not known whether as a homodimer or as a heterodimer. It is likely that ABCG5 and ABCG8 act as heterodimers [Berge et al, 2000. Lee et al, 2001). Other ABC half-transporters, although with a different ABC+TMD arrangement, like TAP1 and TAP2, were also demonstrated to act as heterodimers, when functioning as peptide translocators (Spies et al., 1992).

As ABCG2 is also a half-transporter it is very likely that its active form is a dimer. Several human cancer cell-lines overexpress the ABCG2 multidrug transporter, but it is not known if this protein transports anticancer drugs as a homodimer, or as a heterodimer, interacting with another ABC half-transporter Studies so far utilized mammalian cell lines overexpressing human ABCG2 or its rodent homologue. These cells, however may well contain possible partner proteins of ABCG2 [Knutsen T et al., 2000, Doyle et al., 1998; Litman et al., 2000] Up to the present, no results supporting the existence of homodimers of any of the half transporters have been provided in the art

Thus, despite the evident need for the possibility of testing the interactions of various pharmacological agents and toxic materials with this protein, no screening or testing system comprising ABCG2 free of other, possibly interfering transporters existed in the prior art.

The invention is based on the finding that functional expression of ABCG2 in a heterologous system, particularly in insect cells is possible, indicating that no additional partner protein is required for the activity of this multidrug transporter, which is thus functioning as a homodimer in this system. The inventors provide evidence that ABCG2 expressed in insect cells shows specific, substrate-stimulated ATPase activity. Thus, in this heterologous system the protein can be studied without a possible “physiological” partner. Therefore this assay provides a unique tool for drug testing, without the presence of other endogenous proteins interacting with ABCG2.

Thus, according to an aspect the invention relates to a method for testing drugs for their effect on ABCG2 protein, comprising

providing an ABCG2 protein, preferably of human origin, in an environment essentially free of other, closely related functional ABC transporters,

contacting the provided ABCG2 protein with a drug,

assessing the effect of the drug on the ABCG2 protein.

The environment in which the ABCG2 protein is provided is preferably free of functional ABC transporters of mammalian, more preferably of vertebrata origin. In a further preferred embodiment, the environment is free of ABC transporters.

The environment free of closely related functional ABC transporters is preferably constituted by insect cells or membranes of insect cells.

In an embodiment the ABCG2 protein is expressed in insect cells, preferably in Sf9 cells.

In a preferred embodiment assessing the effect of the drug comprises the steps of i) detecting or measuring at least one type of activity of the ABCG2 protein, ii) comparing the result(s) obtained in step i) with analogous results from a suitable control, iii) evaluating a change in the activity of the ABCG2 protein. Preferably, the type of activity detected or measured is a) ATPase activity or b) transport activity. ATPase activity can be measured by any method suitable for assaying ATPase. Examples for such measurements are e.g. detection of phosphate liberation [e.g. Sarkadi et al (1992)]. In a further embodiment nucleotide trapping is measured using a trapping agent, e.g. Na-orthovanadate, BeFₓ or AlF₄ (see e.g. WO 0210766). In addition, labeling agents, e.g. [α-³²P]-8-azido-ATP can be used for labeling ABCG2 e.g. in isolated membranes. Transport activity can be detected either in whole cells or in isolated membranes, e.g. in membrane vesicles, by detecting uptake of drugs. The skilled person will know a number of methods for measuring ATPase activity or transport activity or partial activities thereof.

In an embodiment, the inhibitory effect of a drug is assessed. In a further embodiment, the drug tested is a drug potentially applicable in transporter protein research, optionally a dye or a substrate.

In particular, ABCG2 can be any of the following ABCG2 protein variants: 482G, 482T and 482R.

According to a further aspect the invention relates to a method for the expression of active ABCG2 in an expression system free of other, closely related functional ABC transporters, preferably in insect cells, comprising expressing a nucleic acid encoding ABCG2, preferably human ABCG2, in insect cells. Preferably, the method comprises the steps of

i) constructing a viral transfer vector operable in insect cells, comprising the cDNA of ABCG2,

ii) generating recombinant viruses using the transfer vector and infecting insect cells with the viruses,

iii) culturing the insect cells to express ABCG2 and optionally

iv) selecting the insect cell clones expressing ABCG2, and/or

v) obtaining the ABCG2 protein, e.g. in the form of membrane preparation.

Preferably, the viruses are baculoviruses.

According to a further aspect the invention relates to an isolated ABCG2 homooligomer, preferably homodimer which are, in a preferred embodiment obtained by expression in insect cells. The invention also relates to uses of the isolated ABCG2 homooligomer for testing drugs.

In a further aspect the invention relates to an insect cell membrane preparation comprising active ABCG2, preferably of human origin, embedded in the membrane. The insect cell membrane preparation preferably comprises active, embedded ABCG2 consisting of membrane vesicles, preferably inside-out membrane vesicles, and more preferably membrane vesicles prepared from Sf9 cells. In the membrane preparation of the invention the ABCG2 is preferably present in a homooligomeric, preferably a homodimeric form. ABCG2 protein can be e.g. any of the following ABCG2 protein variants: 482G, 482T and 482R

In a further embodiment the invention relates to cells, preferably insect cells expressing functional, preferably active ABCG2 protein, said cells being free of other, closely related, functional ABC-transporters.

The invention also relates to reagent kits for testing drugs for their effect on an ABCG2 half transporter protein, comprising at least means for providing an ABCG2 protein in an environment free of closely related functional ABC transporters. The means for providing an ABCG2 protein preferably comprise an isolated nucleic acid encoding ABCG2 protein and at least one means for transfecting said nucleic acid into insect cells and expressing it therein, e.g. suitable restriction enzymes, virus vectors, buffers, media etc. If the ABCG2 protein is to be expressed in insect cell/baculovirus system, the kit may comprise any constituent of usual baculovirus trasfection/expression kits.

According to a further aspect the invention relates to a method for identifying ABCG2 activity in a biological sample, comprising

i) contacting one or more specific activator and/or inhibitor compound of ABCG2 protein with a biological sample possibly comprising ABC transporter activity,

ii) detecting a change in any kind of activity of the ABC transporter caused by each of the compounds relative to a control biological sample which has not been contacted with the respective compound,

iii) evaluating the data obtained in step ii) to decide whether the change in the activity or the pattern of changes in the activity caused by one or more compounds, respectively is characteristic to ABCG2 protein or not.

In a preferred embodiment in detection step ii), above, any of the following compounds and their effect on activity are tested: a) a change in the vanadate sensitive ATPase activity caused by Furnitremorgin C or Verapamil, b) a lack of activation of ATPase activity by Calcein AM, c) an activation of ATPase activity by mitoxantrone. Steps using any of the above compounds can be combined to create a pattern of data of changes more specific to ABCG2. The skilled person will recognized that by using the a method of the invention many further differences in substrate, activator or inhibitor specificity of ABCG2 can be found which are suitable for identifying ABCG2 activity in a biological sample.

Definitions

“ABC transporters” are transporter proteins belonging to the ABC protein superfamily and are capable of; in their native, active, wild type form, extruding drugs from the cells expressing them. Herein, the term “ABC trasporter” also covers mutant variants of the wild type proteins retaining at least one function of the wild type, even if lacking activity.

“Multidrug transporters” are capable of extruding multiple kind of drugs from the cells.

A “half transporter (protein) of the ABCG family” is an ABC transporter which is a product of any of the abcg genes [such proteins are disclosed e.g. on the web-site http://nutrigene.4t.com/humanabc.htm/ and in publications of O'Hare et al., 1984; Pepling and Mount,1990; Dreesen et al., 1988; Tearle et a]., 1989] or a mutant, variant or homologue thereof which retains at least one function of the any wild type form of the protein, preferably an active mutant variant or homologue.

Members of the “ABCG/white subfaiy” are ABC half-transporters which contain one ABC and one TMD domain where the ABC precedes (i.e. N-teminal to) the TMD [see e.g. Berge et al., (2000), Klucken et al., (2000)].

The terms “ABCG2 protein”, “ABCG2” or “ABCG2 transporter protein” are used interchangeably and are meant as a half transporter of the ABCG family belonging to the ABCG/white subfamily, and which is a product of the abcg2 gene or a mutant, variant or homologue thereof which retains at least one function of the any wild type form of the protein, preferably an active mutant variant or homologue. ABCG2 is also termed as MXR Mitoantrone Resistance protein, Miyake et al., 1999.), BCRP (Breast Cancer Resistance Protein, Doyle et al.,1998) or ABCP (placenta specific ABC trasporter, Allikmets et al., 1998). In harmony with the ABC nomenclature, we use the name ABCG2 multidrug transporter throughout the description.

The sequence of the cDNA derived from the abcg2 gene is available from several sources (WO 0136477, Doyle et al. (1998), and http://nutrigene.4t.com/humanabc.htm/). A person skilled in the art understands that sequences of further abcg2 genes (or their cDNA) from further sources will be published in the future and mutant versions will be prepared. These sequences are also appropriate for the purpose of the subject invention, provided that their necessary functions are retained, which can be decided readily by a skilled person based on the present disclosure.

A “function” of a given protein or its fragment is meant as any capability, non-structural feature or property appearing either in vivo or in vitro and characteristic also of any wild type variant. Thus, without any limitation, “functions” are e.g. the following: capability of the protein or its fragment to become glycosylated or folded properly, its targeting, assembly of the protein or participation of a fragment in such an assembly, activity or partial activity.

The term “activity” of ABC transporters is a function involving e.g. the transport of a drug through the membrane carrying the protein, ATPase activity etc. and any partial reaction of the whole reaction cycle of the enzyme (e.g. substrate binding) as well as a partially damaged activity, e.g. nucleotide occlusion (trapping).

The term “isolated” is meant herein as “changed by man compared to its natural environment. If a compound or biological material, e.g. protein or its natural equivalent can be found in nature, than if “isolated”, then it is changed in its original environment or removed from its original environment or both.

A “homooligomer” is a multimeric protein consisting of more than one and less than 32, preferably 16, more preferably 8 identical polypeptide subunit. A special homooligomer is a homodimer consisting of 2 identical subunit.

An ABC transporter “closely related” to ABCG2 is, in a broad sense, an ABC transporter having the same domain structure than ABCG2 and being capable of binding in its active form to ABCG2 and exerting its activity in this assembled form. In particular, the “closely related” ABC transporter can be a protein of vertebrata, more particularly of mammalian origin, or a protein showing a high sequence similarity to ABCG2, e.g. a sequence similarity of 50, 70, 85 or 95%.

### BRIEF DESCRIPTION OF THE FIGURES

FIG. 1a: Immunoblot detection of the ABCG2-multidrug transporter expressed in Sf9 insect cells. Whole cell lysates, dissolved in disaggregation buffer, were subjected to electrophoresis on 10% Laemmli-type gels and blotted to PVDF membranes, followed by immunodetection with anti-MXR 405 antibody, as described in the Methods. Lane 1: MCF-7/MX, 10 μg; lane 2 MCF-7/MX treated with 5 μg/ml tunicamycin, 15 μg; lane 3: ABCG2-expressing Sf9 cells, 1 μg; β-galactosidase-expressing Sf9 cells, 10 μg.

FIG. 1b: Membrane topology model of ABCG2. The numbers indicate the predicted transmembrane helices. Predicted N-glycosylation sites are also indicated.

FIG. 2: Comparison of the effects of various compounds on the vanadate sensitive ATPase activity in isolated Sf9 membranes of ABCG2 (FIG. 2a) or MDR1 (FIG. 2b) expressing Sf9 cells. ATPase activity of isolated Sf9 membranes was determined by measuring vanadate sensitive inorganic phosphate liberation, using 3.3 mM MgATP, as described in the Materials and methods. Data points indicate the mean±S.D. values of at least four measurements, performed in two or three different membrane preparations. Control values show the activity measured in the absence of added compounds. On the plots, the following symbols are applied: (-▾-) for FTC, (-▴-) for CsA, (-♦-) for verapamil, (-▪-) for mitoxantrone, (-X-) for CalceinAM, (-●-) for prazosin, (▪) for control.

FIG. 3a: Effect of cyclosporin A on the basal and the prazosin stimulated ATPase activity in ABCG2 expressing Sf9 cells. CsA concentration was varied at a constant (10 μM) prazosin concentration. The data points show the mean values of at least four determinations. Symbols: (-▾-): CsA+10 μM prazosin, (-▪-): CsA.

FIG. 3b: Effect of prazosin on the ABCG2-ATPase activity in the presence of cyclosporin A. Prazosin concentration was varied at constant (0.5 μM or 2 μM) CsA concentrations. The data points show the mean values of at least four determinations. Symbols: (-▪-); prazosin, (-●-): prazosin+0.5 μM CsA, (-▾-); prazosin+2 μM CsA

FIG. 3c. Effect of FTC on the prazosin stimulated ATPase activity in ABCG2 expressing Sf9 cells. Reciprocal ATPase concentration is given as a function of reciprocal prazosin concentration which was varied at a constant FTC concentration. Symbols: (▪): prazosin, (▾): prazosin+FTC.

FIG. 4. Vanadate-sensitive ATPase activity of ABCG2 variants—effects of prazosin, verapamil and FTC.

FIG. 5. Mitoxantrone uptake in intact Sf9 cells expressing ABCG2 variants. A: K86M mutant, B. 482R variant, C: 482G variant, D: 482T variant.

Flow-cytometry determination of MX fluorescence after 30 min incubation at 37° C.

autofluorescence

20 μM MX+10 μM FTC

20 μM MX

FIG. 6. Hoechst dye uptake followed by fluorescence spectrophotometry in Sf9 cells expressing ABCG2482G (Panel A) or ABCG2 K86M mutant (Panel B). Abscissa: time in seconds. Ordinate: relative fluorescence in arbitrary units.

Below the invention is explained in more detail by non-limiting, illustrative examples. It is to be understood that, besides those explicitly mentioned in the Examples, practically any drug can be studied or tested according to the invention for its effect on ABCG2 transporter protein.

Furthermore, a skilled person will recognize that the expression system used in the examples can be replaced by an other suitable heterologous expression system, preferably an insect cell expression system, more preferably an Sf9/baculovirus expression system. Any modification or improvement on the expression system used is within the scope of the invention provided that ABCG2 is expressed in an environment free of other closely related, functional ABC transporter. Various suitable expression systems are well known in the art. Preferable insect cell expression systems are disclosed in e.g. Galleno, M, Sick, A. J. (1999).

### EXAMPLE 1

### Materials and Methods

Materials—Mitoxantrone, daunorubicin, doxorubicin, prazosin, rhodamine 123, cyclosporinA, verapamil Na-orthovanadate, NEM, calf intestine and alkaline phosphatase were purchased from Sigma, mitoxantrone was obtained from Lederle Laboratories. Fumitremorgin C was provided by Dr. Lee M. Greenberger (Wyeth-Ayerst Research).

Generation of the human ABCG2 containing transfer vector—pAcUW21-L/ABCG2 was constructed by removing the full length human ABCG2 cDNA (G482 variant) [Miyake et al., 1999.] from pcDNA3.1(−)/ABCG2 with SacI digestion, and ligating the resulting fragment to the SacI site of the modified baculovirus transfer vector, pAcUW21-L [Szakacs et al., (2001)].

Additional ABCG2 variants (482R, T and K86M) were created by overlap extension PCR [Morton, RM. (1993) In vitro recombination and mutagenesis of DNA SOEing together tailor-made genes” pp. 251-268]. In each case two internal complementary primer pairs were used, each containing the specific mutation: the primer pairs used for 482 R were 5′ ttattaccaatgcgcatgttaccand and 5′ ggtaacatgcgcattggtaataa, the primer pairs used for 482 T mutant were 5′ ttattacctatgacgatgttacc and 5′ ggtaacatcgtcataggtataa, and the primer pairs used for K86M mutant were 5′ tggaggcatcttcgttatta and 5′ taataacgaagacatgcctcca. The two outer primer pairs used for the 482 variants were 5′ cttgggatacttgaatcagc and 5′ ggtcatgaagtgttgcta and for the K86M mutant 5′ gtatttaaatactatactg and 5′ ggctcatccagaacaagat. The PCR reactions were performed as described in Szaldás et al., (2001). The PCR products containing the 482R or 482T coding sequence were digested with PstI and MscI enzymes and ligated between the corresponding sites of the pAcUW21-L/ABCG2 vector. The PCR product coding the K86M variant was digested with NotI and SpeI enzymes and ligated to the NotI and SpeI sites of the pAcUW21-L/ABCG2 vector. The mutations were confirmed by sequencing the PstI-MscI or the NotI-SpeI fragments of the contstruct, respectively.

Generation of recombinant baculoviruses—Recombinant baculoviruses, carrying the human ABCG2 cDNA were generated with the BaculoGold Transfection Kit (Pharmingene), according to the manufacturer's suggestions. Sf9 cells were infected and cultured as described [Müller et al., (1996)]. Individual clones expressing high levels of the human ABCG2 were obtained by end-point dilution and subsequent amplification. The clone producing the highest yield of the ABCG2 protein was selected by immunoblotting (see below). Sf9 cell membranes were isolated as described [Sarkadi et al., (1992)].

Cell culture and tunicamycin treatment—MCF-7/MX cells [Yang et al., 1995] were cultured in IMEM medium supplemented with 10% FCS, penicillin, streptomycin and 10 nM mitoxantrone at 37° C. in 5% CO2. For inhibition of N-glycosylation the MCF-7/MX cells were grown for 60 hours in a medium containing 5 μg/ml tunicamycin (Sigma).

Membrane preparation, immunoblotting and Coomassie-staining—Three days after virus transfection the Sf9 cells were harvested, their membranes were isolated, and the membrane protein concentrations were determined by the modified Lowry method as described. Proteins of isolated Sf9 membranes were separated on 10% Laemmli-type SDS-gels, the proteins were electro-blotted onto PVDF membranes. Immunoblotting was performed on samples (whole MCF-7 MX or Sf9 cell lysates or Sf9 membrane vesicles) dissolved in disaggregating buffer (DB) [Müller et al, (1996)] using anti-MXR 405 policlonal antibody (2000-folds dilution) [Litman et al., 2000] as first antibody and an anti-rabbit HRP-conjugated secondary antibody (10,000× dilution, Jackson Immunoresearch). HRP-dependent luminescence was developed by the enhanced chemiluminescence technique (ECL, Amersham).

For Coomassie staining the SDS-poliacrilamide gels were incubated in Coomassie Brillant Blue dye overnight.

ATPase activity measurements—Membrane ATPase activity was measured by colorimetric detection of inorganic phosphate liberation as described [Sarkadi et al. (1992)], with minor modifications. The reaction mixture contained 40 mM MOPS-Tris (pH 7.0), 50 MM KCl, 2 mM dithiothreitol, 500 μM EGTA-Tris, 5 mM Na-azide, 1 mM oubain and 5-20 μg membrane protein. The reaction was started with addition of 3.3 mM MgATP. The vanadate sensitive faction was determined in the presence of 1 mM Na-orthovanadate.

Membrane preparation and immunoblotting—Three days after virus transfection, the Sf9 cells were harvested, their membranes were isolated, and the membrane protein concentrations were determined by the modified Lowry method. Proteins of isolated Sf9 membranes were separated on 10% Laemmli-type SDS-gels and the proteins were electro-blotted onto PVDF membranes. Immunoblotting was performed as described earlier [Müller et al., (1996)], by using the anti-MX 405 polyclonal antibody [Litma et al., (2000)], in 2,000× dilution, and an anti-rabbit HRP-conjugated secondary antibody (10,000× dilution, Jackson Immunoresearch). HRP-dependent luminescence was developed by the enhanced chemiluminescence technique (ECL, Amersham).

Mitoxantrone uptake—Functional detection of ABCG2 expression was carried out by measuring MX uptake, based on the method of Robey et al. (2001). Cells were washed once and suspended in HPMI. Aliquots of the suspension containing 3×10⁵ cells were incubated with or without the addition of 5 μM MX, and also with 5 μM MX+10 μM FTC (a specific inhibitor of ABCG2). After an incubation for 30 minutes at 37° C. the cells were washed with ice-cold HPMI medium and than suspended in ice-cold HPMI containing 1.51 μl 10 mg/ml propidium-iodide and stored on ice until the flow cytometry measurements. FACSCalibur cytometer equipped with a 635 nm red diode laser and a 670 nm bandpass filter was used to determine the fluorescence of cellular MX. Events were counted up to 15000 and dead cells were excluded based on propidium iodide staining.

Hoechst33342 uptake—Hoechst dye uptake (5 μM) was measured in a fluorescence spectrophotometer at 350 nm (excitation)/460 nm (emission), by using 3×10⁵ cells in a HPMI solution. This dye becomes fluorescent only in a complex with DNA (Haugland, MP catalogue). The initial increase of fluorescence is due to a rapid dye uptake and nuclear staining in dead cells, while further cellular dye uptake is reflected by an increase in fluorescence. At the end of each experiment, for standardization, a full cellular staining is obtained by the addition of 8 μM digitonin, disrupting the integrity of the cell membrane.

### EXAMPLE 2

### Expression of ABCG2 in Sf9 Cells

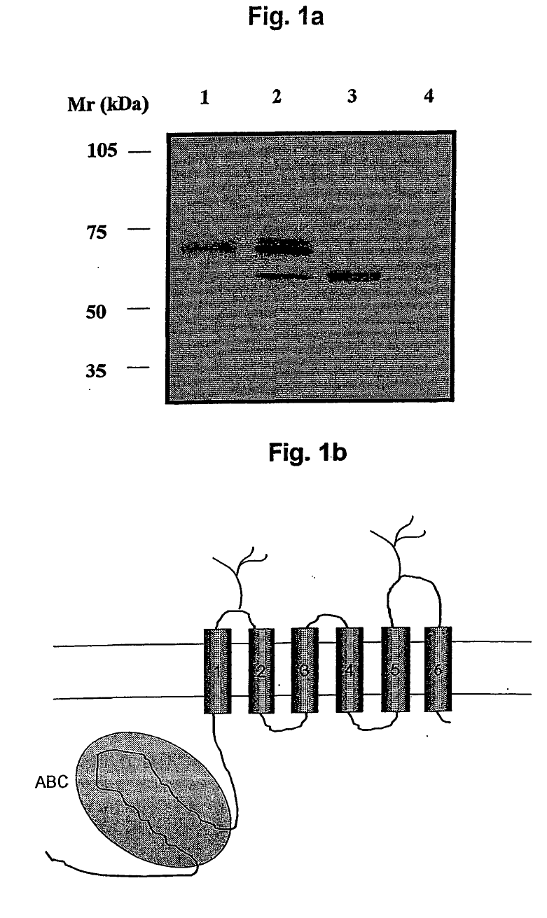

ABCG2 cDNA was cloned into a baculovirus vector and Sf9 insect cells were infected with the recombinant virus. The immunoblot presented in FIG. 1a demonstrates that the ABCG2 protein was efficiently expressed in the baculovirus-infected Sf9 cells. The expression level of ABCG2, as recognized by the MXR-specific antibody, in Sf9 cells (lane 3) was found to be approximately ten times higher than that in the MCF-7/MX mitoxantrone-selected, highly multidrug resistant [Ross et al., (1999), Yang et al., (1995)] breast cancer cells (lane 1). The doublet protein bands, corresponding to the immunoreactive ABCG2 in the Sf9 cell membranes, were also visible by Coomassie-Blue staining of the gels (not shown).

As shown in FIG. 1a, ABCG2 expressed in Sf9 cells migrated at a lower apparent molecular mass (as a doublet around 60 kDa) than the ABCG2 in MCF-7/MX cells (a wide band at about 70 kDa). Membrane proteins expressed in insect cells are underglycosylated [see Bakos et al., (1996), Germann et al., (1990)], which could explain the lower molecular mass of the Sf9-expressed ABCG2-protein. In order to examine this possibility, we have cultured MCF-7/MX cells in the presence of 5 μg/ml tunicamycin, a known inhibitor of N-glycosylation. In samples obtained from tunicamycin treated MCF-7/MX cells (FIG. 1/B, lane 2) two different faster migrating forms of ABCG2 were observed, and the non-glycosylated ABCG2 migrated with a similar apparent molecular mass as the Sf9-expressed protein. In experiments not shown on the figures, the isolated MCF-7/MX cell membranes were treated with N-glycosydase. In this case a strong reduction in the apparent molecular mass of the ABCG2 protein was observed, and the de-glycosylated protein co-migrated with ABCG2, expressed in Sf9 cells [Litman et al., manuscript in preparation].

The membrane topology model of ABCG2 [FIG. 1b—see Miyake et al., (1999) and Allikmets et al., (1998)] predicts two possible N-glycosylation sites. All the above data suggest that both glycosylation sites are active in the mammalian cells, while only a partial, core glycosylation of the ABCG2 is performed by the Sf9 cells. The doublet bands on FIG. 1a, lane 3 probably represent the non-glycosylated and the core-glycosylated forms of ABCG2, respectively, in Sf9 cells. Glycosylation may have a role in the routing or processing of ABCG2. However, it has been convincingly demonstrated that the function of the ABC multidrug transporters MDR1 and MRP1 are unaffected by glycosylation [Schinkel et al., (1993), Gao et al., (1996), Sarkadi et al., (1992), Bakos et al., (1996), Germann et al., (1990)].

### EXAMPLE 3

### ABCG2-Dependent ATPase Activity and its Modulation in Isolated Sf9 Cell Membranes

Multidrug resistance ABC transporters utilize the energy of ATP for their drug transport activity. In the case of MDR1 and MRP proteins both their drug transport activity and the related ATP cleavage are inhibited by Na-orthovanadate, and by SH-group modifying agents, like N-ethylmaleimide (NEM). The function of these transporters, when expressed in Sf9 cells, has been successfully studied by measuring their vanadate-inhibited and substrate-stimulated ATPase activity [Sarkadi et al., (1992), Scarborough (1995), Bakos et al., (2000)]. A membrane ATPase activity, related to the overexpression of ABCG2 in mammalian cells, has already been demonstrated [Nielsen et al., (2000)].

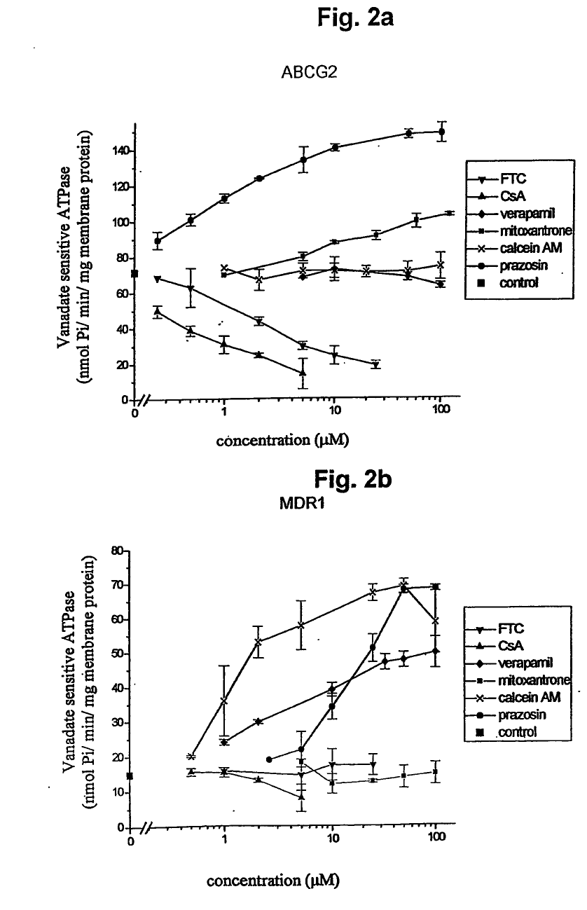

As shown in FIG. 2a, when ABCG2 was expressed in Sf9 cells, in the isolated membranes we observed the appearance of a high capacity (about 70 nanomoles/mg protein/min), vanadate-sensitive ATPase activity (control, meaning DMSO control). Such a basal ATPase activity was absent in control, β-galactosidase expressing membranes (β-gal control), while the vanadate-insensitive membrane ATPase had a similar low-level as found in the β-gal control membranes (not shown). Vanadate-inhibition of the ATPase activity occurred with a Kᵢ value of about 20 μM Na-orthovanadate. NEM also inhibited the ATPase activity at micromolar concentrations (K, NEM was 10 μM—data not shown in detail). The MgATP concentration producing half-maximum membrane ATPase activity was 0.3 mM (see below). All these values for the ABCG2-ATPase are in a similar range as those measured earlier for the MDR1-ATPase activity [Müller et al., (1996), Sarkadi et al., (1992), Homolya et al., (1993)].

### EXAMPLE 4

### Testing of ABCG2 Substrates and Inhibitors

In the following experiments we examined the effects of several, previously described ABCG2 substrates and inhibitors thereby also characterizing the ATPase activity. We have also compared the ATPase activity of ABCG2-containing Sf9 membranes with those containing the human MDR1 protein.

As shown in FIG. 2a, ABCG2-containing membranes had a basal ATPase activity of about 3-5 times higher than that seen in the MDR1-containing membranes (FIG. 2b).

Mitoxantrone (MX)—Addition of mitoxantrone (MX), a well established substrate drug for ABCG2 [Miyake et al., (1999), Ross et al., (1999), Yang et al., (2000), Nielsen et al., (2000)] stimulated the ABCG2-ATPase activity in a concentration dependent manner. In cancer cells MDR1 expression did not evoke significant mitoxantrone resistance [Nakagawa et al., (1992)] and, indeed, mitoxantrone had no significant effect on the MDR1-ATPase activity (FIG. 2/B).

Prazosin—Prazosin, a vasodilatator agent, has been shown to be actively extruded from various multidrug resistant cells [Dey et al., (1997), Litman et al., (2000)]. As shown in FIG. 2, prazosin significantly stimulated the ATPase activity of both ABCG2 and MDR1, although the K_{act} value of prazosin in the case of ABCG2 was about 1 μM, while this value in the case of MDR1 was an order of magnitude higher (about 15 μM).

Verapamil—Verapamil has been shown to be an excellent substrate of MDR1, and it significantly stimulates the MDR1-ATPase activity [Sarkadi et al., (1992)]. In contrast, the multidrug resistance caused by ABCG2 expression was reported to be only slightly sensitive to verapamil [Nielsen et al., (2000) Rabindran et al., (1998)]. In the present experiments we found no verapamil stimulation of the ABCG2-ATPase activity (even a slight inhibition was observed at higher verapamil concentrations—see FIG. 2a). In contrast, as also documented earlier, we observed a 3.3 fold stimulation of the MDR1-ATPase by low concentrations of verapamil (FIG. 2b).

Calcein-AM—Calcein-AM is an excellent MDR1 substrate [Homolya et al., (1993)] and in the present experiments it stimulated the Sf9 membrane MDR1-ATPase 4.5 fold, with a K_{act} of about 1 μM. In contrst, as shown in FIG. 2/A, Calcein-AM had no effect on the ABCG2-ATPase activity. This latter finding is in accordance with results showing no measurable Calcein-AM extrusion from ABCG2 overexpressing, drug-resistant cells [Litman et al., (2000)].

Fumitremorgi C (FTC)—FTC, a fungicide, was described as a powerful inhibitor of the ABCG2-mediated multidrug resistance [Rabindran et al., (1998)] or ATPase activity (Robey et al., Biochim. Biophys. Acta, in press). As shown in FIG. 2, in isolated Sf9 cell membranes Fumitremorgin C strongly inhibited the ABCG2-ATPase, while it had no significant effect either on the basal or the verapamil (33 μM stimulated MDR1-ATPase activity.

Cyclosporin A (CsA)—Cyclosporin A (CsA) has been shown to act only as a weak inhibitor of ABCG2-dependent drug resistance [Doyle et al., (1998)] but decreased the ATPase activity measured in an ABCG2-overexpressing mammalian cell line [Nielsen et al., (2000)]. In the present study we found that CsA inhibited both the ABCG2- and the MDR1-ATPase (see Table I and below).

The kinetic parameters obtained for the effects of the above and some other compounds on the ABCG2-ATPase activity are compiled in Table I. It is important to note that all the above agents had practically no effect on the low level ATPase activity measured in control, β-galactosidase expressing Sf9 cells.

TABLE I
Effects of different drugs on the vanadate sensitive ATPase
activity in membranes of ABCG2-expressing Sf9 cells. Values
in the Table were estimated by the determination of the vanadate
sensitive ABCG2 ATPase activity in two sets of experiment, and
by using at least five different concentrations for each drug.

| | Kact | | Maximum | Maximum |
|---|---|---|---|---|
| Compound | (mM) | Ki (mM) | stimulation (%)* | inhibition (%)* |
| Mitoxantrone | 7 | — | 40 | — |
| Prazosin | 1 | — | 100 | — |
| Doxorubicin | 5 | — | 30 | — |
| Daunorubicin | 2,5 | — | 50 | — |
| Rhodamine 123 | 4,5 | — | 20 | — |
| Cyclosporin A | — | 0,5 | — | 80 |
| Fumitremorgin C | — | 1,3 | — | 74 |

*relative to the ATPase activity measured in the absence of compound

All the above detailed experiments clearly demonstrate that the expression of the human ABCG2 induces a high-capacity membrane ATPase activity, with a distinct substrate-stimulation and inhibitor sensitivity, as compared to those of other human multidrug transporters, like the MDR1 protein. Moreover, the effects of substrates and inhibitors on ABCG2-dependent membrane ATPase activity in all cases showed a close correlation with the effects of these compounds in ABCG2-overexpressing mammalian cells.

In the Sf9 cell membranes the expression levels of the ABCG2 and MDR1 proteins, based on Coomassie-stained gel-electrophoretograms, were found to be similar (not shown), and the maximum, drug-stimulated ATPase activities of the two transporters were also comparable (in the present experiments 140 vs. 70 nanomoles/mg membrane protein/min, for the ABCG2 and the MDR1 protein, respectively). This novel finding indicates tat, similarly to MDR1, ABCG2 is a high-activity, ATP-dependent drug transporter, and its turnover rate greatly exceeds e.g. that of the members of the MRP family [Bakos et al., (2000)].

### EXAMPLE 5

### Characterization of the ABCG2-Expression; Studying the High-Level Basal ATPase Activity in Sf9 Cell Membranes

As shown in FIG. 2, in the ABCG2-expressing Sf9 cell membranes, in contrast to that seen for MDR1, we found a relatively high-level basal ATPase activity. Without being bound by theory, this finding may suggest an endogenous stimulation of the transporter (e.g. by the presence of certain lipids or lipid-derivatives in these membranes), or a partial uncoupling, caused e.g. by the presence of improperly folded ABCG2 molecules.

The idea that a partial uncoupling is a feature of ABCG2 expressed in Sf9 cells is not supported by experiments with a point mutant, K86M of ABCG2. The K86M mutant for ABCG2 has a mutation in a conserved amino acid sequence, thus expected to be non-functional. Indeed, this mutant form had no ATPase activity, showing the close correlaton between transport and ATPase functions.

Therefore the vanadate-sensitive ATPase activity in isolated membranes of Sf9 cells are well applicable for testing drug and modulator interactions with ABCG2.

MgATP-dependence of the ATPase activity—In order to explore the nature of this phenomenon, we measured the MgATP-concentration dependence for the basal and the drug-stimulated ABCG2-ATPase (not shown here in detail). We used 100 μM prazosin as an ABCG2-ATPase stimulating agent. The MgATP-dependence of the ATPase activity under these conditions was similar, although the K_{M}ATP value without an added substrate was 0.6 mM, while in the presence of drug-substrate this value decreased to about 0.3 mM. The Kᵢ value for vanadate inhibition was about 20 μM both in the absence and presence of prazosin. All these data showed a close similarity in the characteristics of the basal and drug-stimulated ABCG2-ATPase activities, and argued against the presence of a misfolded ABCG2 population in the Sf9 cell membrane preparations.

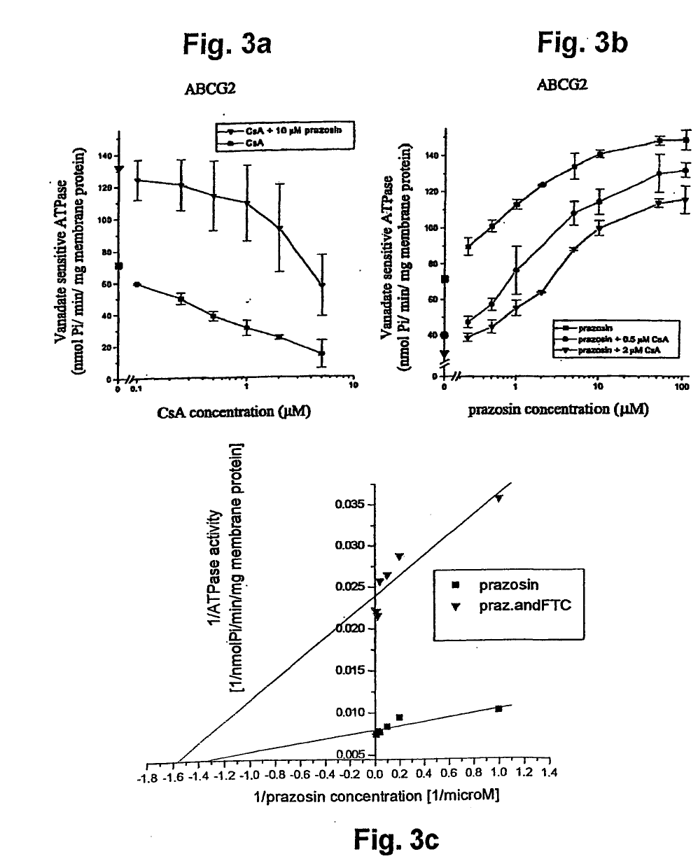

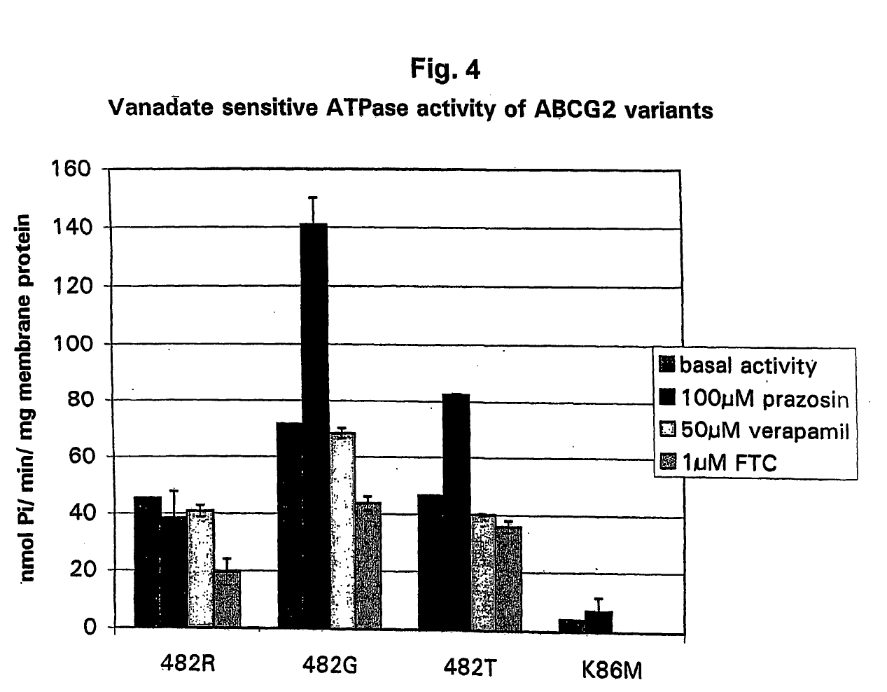

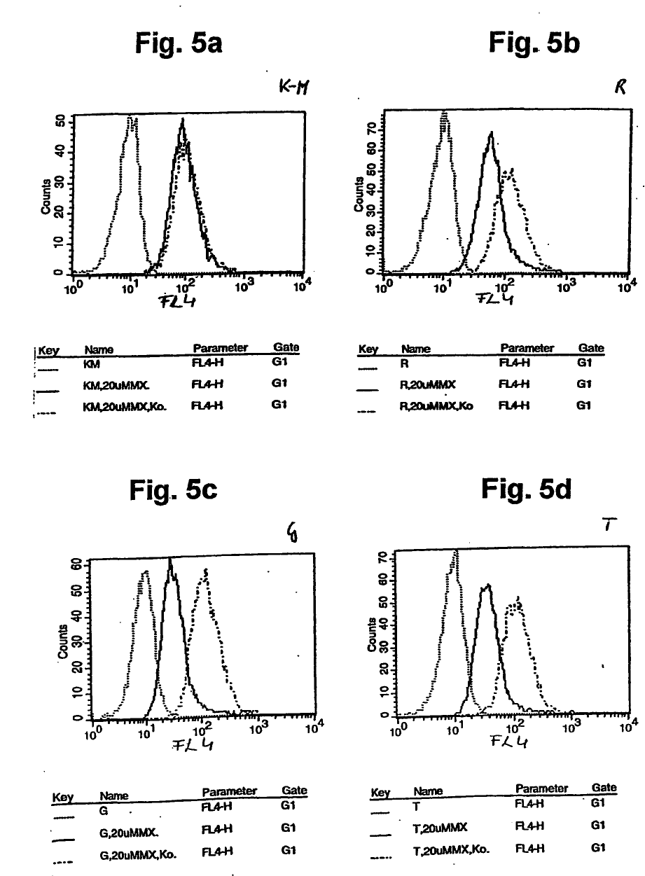

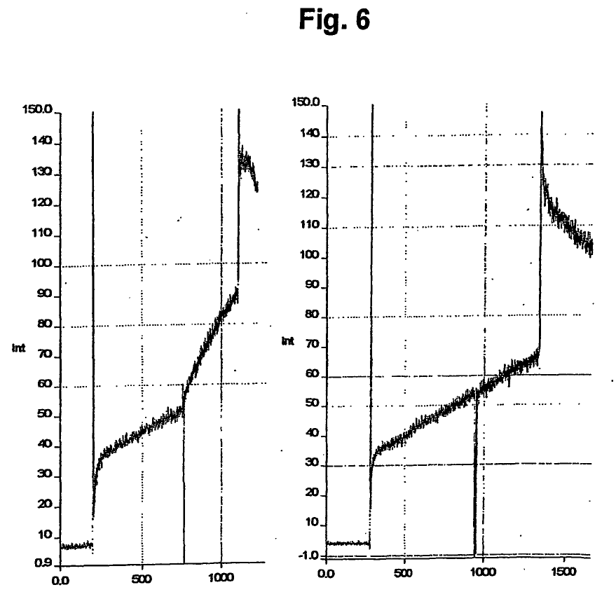

Cyclosporin A (CsA)—In order to further explore the nature of this phenomenon, in the following experiments we measured the ABCG2-ATPase activity in the presence of increasing concentrations of Cyclosporin A (CsA), both in the absence and presence of 10 M prazosin (FIG. 3a). In another set of experiments we varied prazosin concentration in the presence of constant (0.5 or 2 μM) CsA concentrations (FIG. 3b).

We found that CsA inhibited both the basal and the prazosin-stimulated ABCG2-ATPase activity, with K; values of about 0.5 and 1.5 μM, respectively (FIG. 3a). As shown in FIG. 3b, 0.5 or 2 μM Cyclosporin A reduced the basal ATPase activity in ABCG2-containing membranes by about to 45% and 60%, respectively. However, increasing concentrations of prazosin in the presence of CsA still could stimulate the ABCG2-ATPase activity, up to the levels observed in the absence of CsA. There was a significant shift (from 1 μM to about 3 μM in the prazosin concentration producing half-maximum ATPase activation by the presence of 0.5 μM CsA. All these experiments can be interpreted to mean that CsA is a competitive inhibitor of the substrate-stimulated ATPase activity of ABCG2, and the baseline ATPase activity of ABCG2 is induced by a relatively low affinity substrate present in the isolated membranes. This higher baseline activity may explain the relatively smaller magnitude of additional drug-stimulation of ABCG2-ATPase than that found for MDR1.

Various baculovirus clones provide additional proof for existence of homooligomers (homodimers)—The ATPase activity of the membrane preparation derived from Sf9 cells infected with a non clone selected mixture of baculoviruses containing ABCG2 cDNA was also determined. This activity was approximately two-third of that observed in ABCG2-expressing Sf9 membranes, which were derived from Sf9 cells infected with the baculovirus clone showing the highest ABCG2 expression. The ABCG2 expression levels in Sf9 membranes were determined by Western blotting both before and after virus cloning (i.e. selecting of individual clones based on their expression levels). We found that the vanadate sensitive ATPase activity was essentially directly proportional with the level of ABCG2 expression. This observation particularly excludes the possibility that the expression level of a possible dimerisation partner protein of ABCG2 also increased in Sf9 cells infected with the Baculovirus clone giving the highest ABCG2 yields and thereby that activity would necessitate the contribution of such a partner (e.g. an other half transporter). The only possible explanation for the results is that the vanadate sensitive ATPase activity can be attributed exclusively to the presence of the ABCG2 protein. Thus, it is substantiated that ABCG2 forms a homooligomer, or most probably a homodimer.

The functional expression of ABCG2 in this heterologous system indicates that no additional partner protein is required for the activity of this multidrug transporter, which is thus functioning as a homodimer. In this heterologous expression system the presence of a partner ABC half-transporter, with similarly high expression levels can be convincingly excluded. The above results show that the invention provides a unique tool for drug testing, without the presence of possible endogenous proteins interacting with ABCG2.

### EXAMPLE 6

### Whole-Cell Based Functional Tests; Drug Transport Experiments

We have also applied the Sf9 expression system for whole-cell based functional assays of the ABCG2 protein.

Mitoxantrone (MX)—We found that Sf9 cells expressing ABCG2 actively extrude mitoxantrone (MX), which can be determined by flow cytometry. This system allows the determination of drug interactions with the ABCG2 protein, by measuring MX uptake in intact cells. It is important to note that the K86M mutant showed no MX extrusion activity.

Hoechst dye—Another assay applicable for the intact Sf9 cells is the determination of Hoechst dye uptake in intact cells. This dye is a substrate for ABCG2, and its extrusion is characteristic for the function of this protein. The Hoechst dye becomes fluorescent only in a complex with DNA, thus dye uptake and nuclear DNA complex formation is reflected by an increase in fluorescence. Again, we found that Sf9 cells expressing ABCG2 actively extrude the Hoechst dye, which can be determined by fluorescence spectrophotometry. Again, the K86M mutant ABCG2 shows no Hoechst dye extrusion activity. This system allows the determination of drug interactions with the ABCG2 protein, by measuring Hoechst dye accumulation in intact cells.

### EXAMPLE 7

### Application of the Sf9 Cell System for Studying Mutations or Polymorphisms in ABCG2

Sequencing of the abcg2 gene in tumor cells revealed the occurrence of three different ABCG2 transporters, having different amino acids (Arg, Gly or Thr) at position 482. ABCG2-R482 is thought to be the wild-type protein, while the other two variants probably appear during drug selection. In the above experiments we have used the Gly482 variant. The three ABCG2 forms have been recently indicated to show different cross-resistance and drug transport patterns.

In order to apply our test systems for assaying the function of ABCG2 variants, we have expressed several mutant or polymorphic ABCG2 protein variants in Sf9 cells and studied their ATPase and transport characteristics. We found that in isolated membranes all three ABCG2 protein variants (482G, T and R) had a relatively high basal, vanadate-sensitive ATPase activity. However, only the 482G and 482T mutants showed drug-stimulated ATPase activity, when measured in the presence of known ABCG2 substrates.

When measuring the uptake of fluorescent compounds in ABCG2-overexpressing intact Sf9 cells, we found that all the three 482 variants of ABCG2 acted as active extrusion pumps, inhibited by Fumitremorgin C (FTC). When examining the ABCG2 proteins having R. G, or T at position 482, we observed significant differences in their transport activities.

Studying the ATPase activity of these ABCG2 variants, when expressed in Sf9 cells separately or together, should help to investigate different substrate, activator or inhibitor specificities of the variants. Also, possible functional dimerization partners of ABCG2 can be co-expressed and studied in this system. Said dimerization partners can be e.g. different variants or mutants of ABCG2.

Our results presented herein show that the system providing ABCG2 in a related protein free environment, in particular an insect cell membrane ATPase system is an efficient tool for the investigation of the catalytic mechanism of the ABCG2 multidrug transporter, as well as for studying its interactions with pharmacological agents, e.g. anticancer drugs. Since ABCG2 can be studied in itself, false or ambiguous results coming from the interfering effect of other transporters can be excluded. The application of this assay system probably will help significantly the rapid identification of novel substrates and inhibitors of ABCG2 and its variants, with use at the cancer research and clinics.

Besides creating the possibility of testing the effect of drugs on ABCG2 without any possible interfering effect of other ABC transporters, the overexpression test system of the invention provides a more simple and cost effective way of drug testing, than the methods of the art.

### REFERENCES

Allikmets, R., Schriml, L., Hutchinson, A., Romano-Spica, V., and Dean, M. (1998) A human placenta-specific ATP-binding cassette gene (ABCP) on chromosome 4q22 that is involved in multidrug resistance. Cancer Res. 58, 5337-5339.

Al-Shawi, M. K., Urbatsch, I. L., and Senior, A. E. (1994) Characterization of the ATPase activity of purified Chinese hamster P-glycoprotein. J. Biol. Chem. 269, 8986-8992.

Ambudkar, S. V., Dey, S., Hrycyna, C. A., Ramachandra, M., Pastan, I., and Gottesman, M. M. (1999) Biochemical, cellular, and pharmacological aspects of the multidrug transporter. Annu. Rev. Pharmacol. Toxicol. 39, 361-398.

Bakos, E., Hegedüs, T., Holló, Z., Welker, E., Tusnády, G. E., Zaman, G. J., Flens, M. J., Váradi, A., and Sarkadi, B. (1996) Membrane topology and glycosylation of the human multidrug resistance-associated protein. J. Biol. Chem. 271, 12322-12326.

Bakos, É., Evers, R., Sinkó, E., Váradi A., Borst, P., and Sarkadi B. (2000) Interactions of the human multidrug resistance proteins MRP1 and MRP2 with organic anions. Mol Pharmacol. 57, 760-768.

Berge, K. E., Tian, H., Graf, G. A., Yu, L., Grishin, N. V., Schultz, J., Kwiterovich, P., Shan, B., Barnes, R., and Hobbs, H. H. (2000) Accumulation of dietary cholesterol in sitosterolemia caused by mutations in adjacent ABC transporters. Science. 290, 1771-1775.

Borst, P., Evers, R., Kool, M., and Wijnholds, J. (1999) A family of drug transporters: the multidrug resistance-associated proteins. Biochim Biophys. Acta 1461, 347-357.

Dey, S., Ramachandra, M., Pastan, I., Gottesman, M. M., and Ambudkar, S. V. (1997) Evidence for two nonidentical drug-interaction sites in the human P-gycoprotein. Proc. Natl. Acad. Sci. 94, 10594-10599.

Doyle, L. A., Yang, W., Abruzzo, L. V., Krogmann, T., Gao, Y., Risbi A. K., and Ross, D. D. (1998) A multidrug resistance transporter from human MCF-7 breast cancer cells. Proc. Natl. Acad Sci. 95, 15665-15670.

Dreesen, T. D., Johnson, D. H., and Henikoff, S. (1988) The brown protein of Drosophila melanogaster is similar to the white protein and to components of active transport complexes. Mol. Cell Biol. 8, 5206-5215.

Galleno, M. and Sick, A. J. (1999) in: “Gene expression systems” Eds: Fernandez J. M. and Hoeffer J. P., Academic Press

Gao, M., Loe, D. W., Grant, C. E., Cole, S. P., and Deeley, R. G. (1996) Reconstitution of ATP-dependent leukotriene C4 transport by Co-expression of both half-molecules of human multidrug resistance protein in insect cells. J. Biol. Chem. 271, 27782-27787.

Germann, U. A., Willingham, M. C., Pastan, I., and Gottesman, M. M. (1990) Expression of the human multidrug transporter in insect cells by a recombinant baculovirus. Biochemistry 29, 2295-2303.

Homolya, L., Holló, Z., Germann, U. A., Pastan, I., Gottesman, M. M., and Sarkadi, B. (1993) Fluorescent cellular indicators are extruded by the multidrug resistance protein. J. Biol. Chem. 268, 21493-21496.

Horton, R. M. (1993) “In vitro recombination and mutagenesis of DNA SOEing together tailor-made genes” In Methods in Molecular Biology, vol. 15 (PCR Protocols: Current Methods and Applications) (White, B. A., ed.), pp. 251-268, Humana Press, Totowa, N.J.

Jonker, J. W., Smit, J. W., Brinkhuis, R. F., Maliepaard M., Beijnen, J. H., Schellens J. H., Schinkel A. H., (2000) “Role of breast cancer resistance protein in the bioavailability and fetal penetration of topotecan” J. Nat'l Cancer Inst. 92(20), 1651-6.

Klein, I., Sarkadi, B., and Váradi, A. (1999) An inventory of the human ABC proteins. Biochim. Biophys. Acta 1461, 237-262.

Klucken, J., Buchler, C., Orso, E., Kaminski, W. E., Porsch-Ozcurumez, M., Liebisch, G., Kapinsky, M., Diederich, W., Drobnik, W., Dean, M., Allikmets, R., Schmitz, G. “ABCG1 (ABC8), the human homolog of the Drosophila white gene, is a regulator of macrophage cholesterol and phospholipid transport” (2000) Proc Nat'l Acad Sci USA. 97(2), 817-22.

Knutsen, T., Rao, V. K., Ried, T., Mickley, L., Schneider, E., Miyake, K., Ghadimi, B. M., Padilla-Nash, H., Pack, S., Greenberger, L., Cowan, K., Dean, M., Fojo, T., and Bates, S. E. (2000) Amplification of 4q21-q22 and the MXR gene in independently derived mitoxantrone-resistant cell lines. Gene. Chromosome. Canc. 27, 110-116.

Litman, T., Brangi, M., Hudson, E., Fetsch, P., Abati, A., Ross, D. D., Miyake, K., Resau, J. H., and Bates, S. E. (2000) The multidrug-resistant phenotype associated with overexpression of the new ABC half-transporter, MXR (ABCG2). J. Cell Sci. 113, 2011-2021.

Maliepaard, M., van Gastelen, M. A., de Jong, L. A., Pluim, D., van Waardenburg, R. C. A. M., Ruevekamp-Helmers, M., Floot, B. G. J., and Schellens, J. H. M. (1999) Overexpression of the BCRP/MXR/ABCP gene in a topotecan-selected ovarian tumor cell line. Cancer Res. 59, 4559-4563.

Miyake, K., Mickley, L., Litman, T., Zhan, Z., Robey, R., Cristensen, B., Brangi, M., Greenberger, L., Dean, M., Fojo, T., and Bates, S. E. (1999) Molecular cloning of cDNAs which are highly overexpressed in mitoxantrone-resistant cells: demonstration of homology to ABC transport genes. Cancer Res. 59, 8-13.

Müller, M., Bakos, É., Welker, E., Váradi, A., Germann, U. A., Gottesman, M. M., Morse, B. S., Roninson, I. B., and Sarkadi. B. (1996) Altered drug-stimulated ATPase activity in mutants of the human multidrug resistance protein. J. Biol. Chem. 271, 1877-1883.

Nakagawa, M., Schneider, E., Dixon, K. H., Horton, J., Kelley, K., Morrow, C., and Cowan, K. H. (1992) Reduced intracellular drug accumulation in the absence of P-glycoprotein (mdr1) overexpression in mitoxantrone-resistant human MCF-7 breast cancer cells. Cancer Res. 52, 6175-6181.

Nielsen, D., Eriksen, J., Maare, C., Litman, T., Kjaersgaard, E., Plesner, T., Friche, E., and Skovsgaard, T. (2000) Characterisation of non-P-glycoprotein multidrug-resistant Ehrlich ascites tumour cells selected for resistance to mitoxantrone. Biochem. Pharmacol. 60, 363-370.

Pepling, M., Mount, S. M. “Sequence of a cDNA from the Drosophila melanogaster white gene” (1990) Nucleic Acids Res. 18(6), 1633

Rabindran, S. K., He, H., Singh, M., Brown, E., Collins, K. I., Annable, T., and Greenberger, L. M. (1998) Reversal of a novel multidrug resistance mechanism in human colon carcinoma cells by fumitremorgin C. Cancer Res. 58, 5850-5858.

Rao, U. S., and Scarborough, G. A. (1994) Direct demonstration of high affinity interactions of immunosuppressant drugs with the drug binding site of the human P-glycoprotein. Mol Pharmacol. 45, 773-776.

Ross, D. D., Karp, J. E., Chen, T. T., and Doyle, L. A. (2000) Expression of breast cancer resistance protein in blast cells from patients with acute leukemia. Blood 96, 365-368.

Ross, D. D., Yang, W., Abruzzo, L. V., Dalton, W. S., Schneider, E., Lage, H., Dietel, M., Greenberger, L., Cole, S. P., and Doyle, L. A. Atypical multidrug resistance: breast cancer resistance protein messenger RNA expression in mitoxantrone-selected cell lines. (1999) J. Natl. Cancer 191, 429-433.

Sarkadi, B., Price, E. M., Boucher, R. C., Germann, U. A., and Scarborough, G. A. (1992) Expression of the human multidrug resistance cDNA in insect cells generates a high activity drug-stimulated membrane ATPase. J. Biol. Chem. 267,4854-4858.

Scarborough, G. A. (1995) Drug-stimulated ATPase activity of the human P-glycoprotein. J. Bioenerg. Biomembr. 27, 3741.

Schinkel, A. H., Kemp, S., Dolle, M., Rudenko, G., and Wagenaar, E. (1993) N-glycosylation and deletion mutants of the human MDR1 P-glycoprotein. J Biol. Chem. 268, 7474-7481.

Spies, T., Cerundolo, V., Colonna, M., Cresswell, P., Townsend, A, and DeMars, R (1992) Presentation of viral antigen by MHC class I molecules is dependent on a putative peptide transporter heterodimer. Nature 355, 644-646.

Szakács, G., Özvegy, C., Bakos, É., Sarkadi, B, and Váradi, A. (2001) Role of glycine-534 and glycine-1179 of human multidrug resistance protein (MDR1) in drug-mediated control of ATP hydrolysis. Biochem. J. 356, 71-75.

Tearle, R. G., Belote, J. M., McKeown, M., Baker, B. S., Howells, A. J. (1989) “Cloning and characterization of the scarlet gene of Drosophila melanogaster” Genetics 122(3) 595-606.

Yang, C. J., Schneider, E., Kuo, M. L., Volk, E. L., Rocchi, E., and Chen, Y. C. (2000) BCRP/MXR/ABCP expression in topotecan-resistant human breast carcinoma cells. Biochem. Pharmacol. 60, 831-837.

Yang, C. J., Horton, J. K., Cowan, K. H., and Schneider, E. Y. (1995) Cross-resistance to camptothecin analogues in a mitoxantrone-resistant human breast carcinoma cell line is not due to DNA topoisomerase I alterations. Cancer Res. 55,4004-4009.

Abbreviations

MXR, Mitoxantrone Resistance-associated protein; BCRP, Breast Cancer Resistance Protein; ABCP, Placenta specific ABC transporter; ABC, ATP Binding Cassette; Sf9 cells, Spodoptera frugiperda ovarian cells; MDR1, Multidrug Resistance protein; MRP1, Multidrug Resistance-associated Protein; TMD, transmembrane domain; TAP, transporter associated with antigen processing; Calcein-AM, calcein acetoxy-methylesther; NEM, N-ethylmaleimide; MX, mitoxantrone; FTC, Fumitremorgin C; CsA, cyclosporin A.

1. Method for testing drugs for their effect on ABCG2 protein, comprising
expressing an ABCG2 protein in insect cells,
providing the ABCG2 protein having expressed in insect cells in an environment constituted by insect cells or insect cell membranes, preferably membrane vesicles,
contacting the expressed ABCG2 protein with a drug,
assessing the effect of the drug on the ABCG2 protein.

2. A method of claim 1 wherein the ABCG2 protein is of human origin and the insect cells are Sf9 cells.

3. A method of claim 1 or 2 wherein assessing the effect of the drug comprises the steps of
i) detecting or measuring at least one type of activity of the ABCG2 protein, preferably a) ATPase activity or b) transport activity)
ii) comparing the result(s) obtained in step i) with analogous results from a suitable control,
iii) evaluating a change in the activity of the ABCG2 protein.

4. Isolated ABCG2 homooligomer, preferably homodimer said homooligomer or homodimer preferably being obtained by expression in insect cells.

5. Use of an isolated ABCG2 homooligomer of claim 4 for testing drugs.

6. Insect cell membrane preparation comprising active ABCG2, preferably of human origin, embedded in the membrane.

7. An insect cell membrane preparation of claim 6, said preparation comprising active, embedded ABCG2 consists of membrane vesicles, preferably inside-out membrane vesicles, more preferably membrane vesicles prepared from Sf9 cells.

8. Insect cell comprising active ABCG2.

9. Reagent kit for testing drugs for their effect on an ABCG2 half transporter protein, comprising at least means for expressing an ABCG2 protein in insect cells, preferably a nucleic acid encoding ABCG2 protein, and a vector for introducing said nucleic acid into the insect cells and expressing it therein.

10. A method for identifying ABCG2 activity in a biological sample, comprising
i) contacting (a) a substrate and/or (b) an inhibitor with a biological sample comprising ABC transporter activity,
ii) detecting the following effect(s) relative to a control biological sample which has not been contacted with the respective compound,
(a) stimulation and/or inhibition of ATPase activity by a transported substrate and/or
(b) transport of a fluorescent dye and optionally
(c) one or more of the following:
an inhibition of the vanadate sensitive ATPase activity and/or fluorescent dye transport caused by a specific inhibitor of ABCG2, and a lack of effect of a non-ABCG2-interacting agent,
iii) evaluating the data obtained in step ii) to decide whether the effect(s) caused by one or more compounds, respectively is/are characteristic to ABCG2 protein or not.

11. A method of claim 10 for identifying ABCG2 activity in a biological sample, comprising
i) contacting (a) mitoxantrone and/or (b) Hoechst33342, and optionally (c) one or more of the following: Fumitremorgin C, Verapamil and Calcein AM with a biological sample possibly comprising ABC transporter activity,
ii) detecting the following effect(s) relative to a control biological sample which has not been contacted with the respective compound,
(a) stimulation of ATPase activity by mitoxantrone, and/or
(b) Hoechst33342 extrusion, and optionally
(c) one or more of the following:
an inhibition of the vanadate sensitive ATPase activity caused by Fumitremorgin C or
Verapamil and a lack of activation of ATPase activity by Calcein AM,
iii) evaluating the data obtained in step ii) to decide whether the effect(s) caused by one or more compounds, respectively is/are characteristic to ABCG2 protein or not.


SEQUENCE LISTING (10 sequences)

SEQ ID NO: 1 - DNA, 23 bp, Artificial
  Synthetic primer for ABCG2 variant with 482 R mutation
ttattaccaatgcgcatgttacc

SEQ ID NO: 2 - DNA, 23 bp, Artificial
  Synthetic primer for ABCG2 variant with 482 R mutation
ggtaacatgcgcattggtaataa

SEQ ID NO: 3 - DNA, 23 bp, Artificial
  Synthetic primer for ABCG2 variant with 482 T mutation
ttattacctatgacgatgttacc

SEQ ID NO: 4 - DNA, 23 bp, Artificial
  Synthetic primer for ABCG2 variant with 482 T mutation
ggtaacatcgtcataggtaataa

SEQ ID NO: 5 - DNA, 22 bp, Artificial
  Synthetic primer for ABCG2 variant with K86M mutation
tggaggcatgtcttcgttatta

SEQ ID NO: 6 - DNA, 22 bp, Artificial
  Synthetic primer for ABCG2 variant with K86M mutation
taataacgaagacatgcctcca

SEQ ID NO: 7 - DNA, 20 bp, Artificial
  Synthetic outer primer for expression of the 482 mutant ABCG2 protein
cttgggatacttgaatcagc

SEQ ID NO: 8 - DNA, 20 bp, Artificial
  Synthetic outer primer for expression of the 482 mutant ABCG2 protein
ggtcatgagaagtgttgcta

SEQ ID NO: 9 - DNA, 25 bp, Artificial
  Synthetic outer primer for the K86M mutant ABCG2 protein
gtatattaattaaaatactatactg

SEQ ID NO: 10 - DNA, 20 bp, Artificial
  Synthetic outer primer for the K86M mutant ABCG2 protein
ggctcatccaagaacaagat